(19) United States
(12) Patent Application Publication
(10) Pub. No.: US 2022/0280679 A1
(43) Pub. Date: Sep. 8, 2022

(54) Prophylactic Gel Compositions and Use as Barriers to Bacterial and Viral Colonization

(71) Applicant: Innovation Chemical and Enviromental Technologies, Inc., Norwood, MA (US)

(72) Inventor: Shantha SARANGAPANI, East Walpole, MA (US)

(21) Appl. No.: 17/689,148

(22) Filed: Mar. 8, 2022

Related U.S. Application Data
(60) Provisional application No. 63/158,329, filed on Mar. 8, 2021.

(51) Int. Cl. A61L 15/22; A61L 15/44; A61L 31/04; A61L 31/16; A61L 31/14

(52) U.S. Cl. CPC A61L 15/22; A61L 15/44; A61L 31/04; A61L 31/16; A61L 31/145; A61L 2300/404

(57) ABSTRACT

Gel compositions for use as a barrier for microbial attachment and growth on a surface are provided. The gel compositions can prevent crystal formation, for example, calcium-based stones in biological systems. The gel compositions can enhance function of mucosal barriers against microbial adhesion. Specific compositions for anti-bacterial and anti-viral use are provided.

### CROSS-REFERENCE TO RELATED APPLICATIONS

This application claims priority to U.S. Provisional Application No. 63/158,329, filed 8 Mar. 2021, entitled “Prophylactic Gel Compositions and Use as Barriers to Bacterial and Viral Colonization”, the entirety of which is incorporated herein by reference.

### BACKGROUND

Antimicrobial resistance is a global public health challenge, which has accelerated by the overuse of antibiotics worldwide. Overprescribing of antibiotics is associated with an increased risk of adverse effects, more frequent re-attendance, and increased medicalization of self-limiting conditions. Biofilms can be difficult to treat using standard antibiotic therapy, and there are inadequate or no medications available to treat many viral infections. There is a need to develop new products for treating bacterial and viral infections.

### SUMMARY

The present technology provides novel infection control compositions for living surfaces and for inanimate surfaces, for controlling microbial (e.g., viral, bacterial, and yeast or fungus) attachment and replication. The technology provides other advantages such as enhancement of the natural barrier functions of skin and mucosal surfaces and control of crystal growth, for example, calcium-based stones in biological systems. The technology also provides methods for incorporating components with single or multiple functional features into a user-friendly configuration.

The technology presents methods and novel compositions for creating instantly makeable gels (a semi-solid), by adding a water medium or a non-water medium, in situ, at room temperature, to pre-measured components in a in a kit, thereby providing a precise, predictable consistency and lubricity at ambient room temperature.

The technology presents methods and novel compositions for providing topical gels, creams, ointments, patches, and liquid sprays. The technology presents methods and novel compositions for coating devices or textiles such as masks and protective gear activated by moisture. These instant lubricious, semi-solid compositions can be applied on medical devices such as catheters, surfaces and topically on mucosal tissue or skin as appropriate.

The present technology can be further summarized by the following list of features.

1. A gel composition for use as a barrier for microbial attachment and growth on a surface, crystal formation, and/or enhancement of mucosal barrier function, the composition comprising one or more water soluble anionic polymers at a concentration up to 2% w/w of the final gel, one or more gel forming polymers at a concentration up to 3% w/w of the gel, one or more live stabilized probiotic powders at a concentration not exceeding 200 billion cfu/gm of the gel, one or more non-live probiotic fermentates or derivatives at a concentration up to 11% w/w of the gel, one or more natural or synthetic surfactants at a concentration up to 0.5% w/w of the gel, potentiating antioxidants at up to 0.3% w/w of the gel, and/or one or more natural disaccharides, oligosaccharides, or polysaccharides, carbohydrates, sugar alcohols, as growth promoters at up to 0.5% w/w of the gel.

2. The composition of feature 1 that is prepared as a quick-setting gel at room zo temperature, providing a semi-rigid gel like consistency within 10-30 minutes.

3. The composition of feature 1 or 2 that is prepared by dispersing the probiotic powder in a liquid medium followed by addition of a gelling agent comprising said gel forming polymer.

4. The composition of any of the preceding features that is suitable for application to a medical device, such as a catheter, or for application to skin or a mucosal surface of a body.

5. The composition of any of the preceding features which is in the form of a water-soluble gel, ointment, emulsion, foam, or cream.

6. The composition of any of the preceding features, which is 3D printed in form of a pattern or design.

7. The composition of any of the preceding features, wherein the gel forming polymer is selected from cellulose polymers, uncrosslinked polyvinyalcohol, cellulose-based polymers such as carboxy methyl cellulose, hydroxypropyl methylcellulose, polyacrylic acid polymers, plant-based polysaccharides, and combinations thereof.

8. The composition of any of the preceding features, wherein the water-soluble anionic polymer is selected from sulfonated and carboxylated polymers that inhibit crystal formation of insoluble calcium or magnesium salts.

9. The water-based composition of feature 8, wherein the anionic water-soluble polymer provides a mucosal protective effect; and the water-based composition further comprising growth promoters for Lactobacillus comprising sugar alcohols including Mannitol, carbohydrates including D-Mannose, Sugars, or a combination thereof.

10. The composition of any of the preceding features, further comprising a non-aqueous gel carrier selected from silicone-based oils, polymers, and biocompatible non-aqueous carriers such as oil, petroleum-based products, or lipids.

11. The composition of any of the preceding features, wherein said probiotic powder has certified shelf life and potency, and viability assurance.

12. The composition of any of the preceding features, wherein the composition is capable of serving as a barrier for attachment and growth of microbes selected from bacteria, yeast, and viral pathogens.

13. The composition of any of the preceding features, wherein the composition is anti-inflammatory.

14. A dry patch or printed textile, wherein the gel composition of any of the preceding features is formed in or on the patch or textile upon contact with water.

15. A kit of parts comprising two or more pre-packaged components capable of forming a gel, cream, or ointment comprising the gel composition of any of features 1-13 when the components are combined.

16. A kit of parts comprising two or more components placed in pods or capsules, wherein the components are capable of forming the gel composition of any of features 1-13 upon the addition of water.

17. A method for preparing a prophylactic gel, ointment, or cream composition, the composition suitable for use as a barrier for microbial attachment and growth, crystal formation, and/or enhancement of mucosal barrier function, the method comprising combining one or more hydrophilic muco-adhesive agents and one or more non-aqueous carriers.

18. A method to aid in treating or preventing an infection, the method comprising administering to s subject in need thereof the gel composition of any of features 1-13.

19. The method of feature 18, comprising administering the gel composition via a soluble suppository or pellet.

20. The method of feature 18, comprising implanting in the subject's body a medical device comprising or consisting of the composition of any of features 1-13.

21. The method of any of features 18-20, wherein binding of a virus, bacterium, or yeast to a mucosal surface or a cell of the subject is prevented or inhibited.

22. A kit for providing a gel composition, the kit comprising: a capsule, pod, or sachet holding a live probiotic powder; a dispensing container holding a paste comprising hydroxypropyl methylcellulose, phosphate buffered saline powder, glycerin, and simethicone; and instructions for use.

23. The kit of feature 22, wherein the live probiotic powder comprises Lactobacillus rhamnosus belonging to the family of Lactobacilli casei family.

24. The kit of feature 22, wherein the dispensing container is a sterilized syringe, a squeeze tube, or a pouch.

25. A method of providing a gel composition, the method comprising the steps: providing a capsule, pod, or sachet holding a live probiotic powder; providing a dispensing container holding a paste comprising hydroxypropyl methylcellulose, phosphate buffered saline powder 10× concentration, glycerin, and simethicone; dispersing the live probiotic powder into an aliquot of sterile water, and shaking the mixture for about 30 seconds; adding the paste to the aliquot of sterile water; and waiting a time of about 20 minutes for the gel composition to fully hydrate.

26. The method of feature 25 or the kit of feature 22, wherein the paste comprises about 18.4% w/w hydroxypropyl methylcellulose, about 4.5% w/w phosphate buffered saline powder 10× concentration, about 76.7% w/w glycerin, and about 0.3% w/w simethicone.

27. A nasal/facial gel composition comprising hydroxypropyl methylcellulose, lactic acid, polyvinyl sulfonate, glyceryl monolaurate, F127 polaxamer, glycerin, polyethylene glycol, and pH 6 buffer in water.

28. The composition of feature 27, wherein the composition comprises about 1.5%-2% w/w hydroxypropyl methylcellulose, about 0.2%-0.4% w/w lactic acid, about 0.5%-2% w/w polyvinyl sulfonate, about 0.01%-0.3% w/w glyceryl monolaurate, about 0.05%-0.2% w/w F127 polaxamer, about 0.2%-0.7% w/w glycerin, about 0.1%-0.5% w/w polyethylene glycol, and the remaining weight % of pH 6 buffer in water.

29. The composition of feature 28 that provides greater than a 95% reduction in MDCK cell line death by a Human Coronavirus OC-43 infection after a pretreatment of the MDCK cells with the composition for about 2 hours.

30. The composition of feature 28 that provides greater than a 95% reduction in MDCK cell line death by a Human Influenza A H1N1 infection after a pretreatment of the MDCK cells with the composition for about 2 hours.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a zo composition or in a description of elements of a device, can be exchanged with the alternative expression “consisting of” or “consisting essentially of”.

### BRIEF DESCRIPTION OF THE DRAWINGS

FIG. 1 shows an example of a two-part, instant gel single-use configuration such as for a catheter lubricant, as a general direct mucosal surface lubricant, or as a topical gel.

FIG. 2A shows an example of a gel polymer paste or a component release from a blister at the bottom of a container to which liquid (e.g., water and/or probiotic formulation) is added and mixed. FIG. 2B shows an example of components, dry powders, or probiotic compositions packaged in sachets, capsules, or pods, with medically approved dry, swellable hydrophilic polymers.

FIG. 3A shows MTT assay assessing viability of human bladder tissue explants after 24 h treatment with 10 μL doses of ICET UT20 58 product compositions. Active compositions are compared against a commercial control (UT20 58-164-4), a non-irritative control (PBS, phosphate buffered saline), a mild irritant control (1% TX-100, Triton X-100 surfactant). FIG. 3B shows same experimental design as FIG. 3A except tissue explants are pre-treated with 10 μL lipopolysaccharide (LPS) for 1 h. FIG. 3C shows same experimental design as FIG. 3A except human bladder tissue was replaced with porcine vaginal tissue. FIG. 3D shows same experimental design as FIG. 3B except human bladder tissue was replaced with porcine vaginal tissue.

FIG. 4 shows a photo of catheter tips with IC-2B1-type gel placed on E. coli (ATCC 700928); the inoculated plate (55, left) shows a zone of a clearing where the gel was applied in contrast to the control commercial gel (60, right), which shows no clearing.

FIG. 5A shows a biocompatibility MTT assay, histology, and reduction of inflammatory biomarkers upon the application of the iCET compositions, of 1 h LPS inflamed, 10 μL 24 h treatment MTT on porcine nasal tissue. FIG. 5B shows the biocompatibility MTT assay, histology, and reduction of inflammatory biomarkers upon the application of the iCET compositions, of no LPS inflamed, 10 μL 24 h treatment MTT on porcine nasal tissue.

### DETAILED DESCRIPTION

The following functional aspects are provided in the invented compositions a) reduction or inhibition of microbial adherence to surfaces in a contaminated environment b) inflammation reduction c) inhibition of insoluble calcification or calcium stones, d) urease inhibition e) mucosal barrier enhancement and f) inhibition of viral or bacterial attachment to cells.

The compositions can be provided in a ready to use, user-friendly configuration. Pre-measured components can be provided in a kit for compositions, liquids, coatings, gels, creams, ointments, patches, and liquid sprays. In each composition, each component is selected with a functional application, and optional additives, formulations, or optional lubricants such as glycerin, propylene glycol, aloe vera can be added to provide the final compositions. With a process of combining the components to meet specific requirements, the technology provides the use of one or more of the four functional components shown below in tailor-made compositions without preservatives as aqueous or non-aqueous compositions.

Component 1. One or more active components with anti-microbial, bacteriostatic or bactericidal property, with examples “a-f” below:
a) such as a from a probiotic family of live organisms as dry stable powders with or without prebiotic additives such as sold commercially in capsules, sachets, or as bulk probiotic powders with indicated shelf life.
b) a suitably bulk encapsulated or microencapsulated in a biocompatible water-soluble shell.
c) a product derived from probiotic fermentation and non-live probiotics, such as heat killed probiotic.
d) a non-probiotic antimicrobial such as a natural surfactant, for example, glyceryl monolaurate, curcumin, basil turmeric extract and the like.
e) Potentiating antioxidants such as Vitamin C as ascorbate, alpha-hydroxy acids, aminocarboxylates, and sugars.
f) Probiotic growth promoters such as small amounts of sugars, sugar alcohols with osmotic diuretic property such as mannitol carbohydrates, such as D-mannose, and substrates that would facilitate the increased growth rate of Lactobacillus probiotics while competing with pathogenic bacteria.

Component 2. An instantly viscosity increasing synthetic or natural polymer that is hydrophilic with or without prebiotic or mucoadhesive property, as such polymers are expected to synergistically enhance mucoadhesion of the poly anions and the antimicrobials.

Component 3. A synthetic mucosal glycosaminoglycan (GAG) enhancing component with crystal inhibition properties and viral replication inhibition properties.

Component 4. Passive and active barriers for viral and bacterial pathogens (topical and nasal).

To achieve one or more of the functional aspects above, examples of each component of the compositions is described in more detail, then the compositions are described and tested.

Example Component 1 includes probiotic powders stabilized with certified potencies and viability assurance. For administration of this component, a local delivery of probiotics is more advantageous as it reduces the gastrointestinal exposure and potential side effects of oral probiotics.

The technology presents a locally applied, weakly reversible mucoadhesive containing probiotic gel, which with daily use will allow continued levels of Lactobacillus at the mucosa surface, blocking the adherence of pathogens and could thus enhance the mucosal barrier properties.

Lactobacilli are known to exhibit antimicrobial, anti-inflammatory properties and to interfere with the spread of pathogenic organisms within the colonized part. However, for local delivery the viability needs to be consistent at the time of use. Therefore, premade gels with probiotics with common water-soluble lubricant gel materials requiring water would compromise the quality and performance due to the lowering of viability during production, storage. Shelf-life issues can arise. To overcome this, a user-friendly instant gel composition is developed and demonstrated in this technology. However, certain encapsulated, stabilized versions of Lactobacillus and non-aqueous media such as silicone oil or lipids based premade compositions are also the result of this technology.

This component also includes probiotic derived materials as a component antimicrobial. A Lactobacillus fermentate, for example, Leucidal™ commercially sold as cosmetic preservatives (Lotion Crafter, WA, sourced from Active microtechnologies) can be found useful in this technology in exhibiting both antimicrobial and urease inhibiting properties, as active ingredients that provide or enhance bacteriostatic or bactericidal action.

Example Component 2 includes a gelling polymer component with mucoadhesive or film-forming properties. This component provides a means to increase the viscosity, for example, to create creams or gels with or without mucoadhesive properties. The same can be used for encapsulation (if a film or coating is desired) and can serve as a prebiotic if probiotics are used. It comprises synthetic or natural polymers known in the art for enhancing the non-covalent, temporary, mucoadhesive delivery of the components (e.g., hypromellose or HPMC—hydroxypropyl methylcellulose of various viscosities and molecular weights), and for prolonging contact time with mucosa.

The adhesion of certain polymers to mucosal surfaces is through non-covalent interactions such as weak van der Waals forces, hydrogen bonding, electrostatic attraction, hydrophobic effects, diffusion, and interpenetration (entanglements), which are seen in polymers with ionic groups such as sulfate, carboxylate, etc. The cellulose polymers, for example, extensively used in lubricants and drug delivery are not strongly mucoadhesive. However, due to the highly loose hydrophilic network, can allow longer contact time for Lactobacillus cells to populate the mucosal surface and colonize. Hydroxypropyl methylcellulose has thermal gelation properties, and other similar medical grade polymers are applicable for this component.

In addition, medically used polymer vehicles such as Carbomer™ based polymers, all types of Polaxamer polymers (Pluronics), polyethylene glycols, polyethylene oxide, sodium carboxyl-methyl cellulose (CMC), polyvinyl alcohol, polyvinyl pyrrolidone and the like also are compatible with this component.

Pre-hydrated forms, or use of a non-solvent such as glycerol or polymeric glycols prior to addition of water can help dispersion. For example, Pre-Hydrated Ticalose® CMC 6000 is a high viscosity agglomerated sodium carboxymethylcellulose derived from cellulose. It is soluble in both cold and hot water, stable over a pH range of 4.0-10.0, improves cold water dispersibility and hydration. Pre-Hydrated Ticalose CMC 6000 usage level is about 0.075% to about 2%.

Natural gels such as pectin and gums such as hyalurate based, Aloe vera, seed mucilages, such as from basil seeds, holy basil seeds, chia, and botanically derived hydrophilic materials can be used.

Films or coatings can comprise polysaccharides and proteins. Examples of the polysaccharides are cellulose derivatives, dextrans, inulin, alginate, carrageenan, starch derivatives, pectin derivatives, chitosan, seaweed extracts, and galactomannan. A commonly used polysaccharide is alginate. Alginates possess good film-forming properties and produce transparent and water-soluble film.

Medical grade or food grade non-cross linked polyvinyl alcohol that is cold water-soluble films or sachets are of high utility here. Each individual coating material possesses some unique, but limited functions and a combination of different materials can be more effective.

Example Component 3 can provide mucosal barrier enhancement by poly anionic materials which can have antiviral properties and prevent nucleation of calcium magnesium stone crystal formation. Certain polyanionic materials also provide inhibition of viral attachment to cells and could also serve as a mucosal enhancement material, such as heparin.

Mucous membranes that line various orifices of the body generally are hydrophilic as they contain many macromolecules in the mucin (gel-like) layer with a large amount of water within its composition. The mucin is composed of compounds called glycosaminoglycan (GAG) that enable the formation of a gel-like substance and enhance the mucosal barrier. An anionic synthetic polymer, polyvinyl sulfonate (PVS) component, is predicted to be a GAG (glycosaminoglycan) enhancing, anti-adhesive, weak Heparin mimic and a potent calcium stone inhibitor. The PVS is anticipated to enhance the glycosaminoglycan layer and discourage bacterial adherence on the mucosal surface (as observed for heparin and some sulfonate bearing biopolymers). Polyanionic materials natural and synthetic are useful in this component.

Example Component 4 can provide passive and active barriers for viral and bacterial pathogens (e.g., topical and nasal). Respiratory pathogens are spread mainly by droplets made when people cough, sneeze or talk. These droplets may contaminate the face and nostrils and can possibly be inhaled into the lungs. The mucus layer entraps particles, which are then cleared from the nasal cavity by the cilia.

With current pandemic era and viral mutants and bacterial resistance, it is envisioned that component can also be used as an ingredient in topical gel/cream or wipes for the nostrils, nose and surrounding area, face, neck, and possibly hands.

A composition with this component can provide a first line of defense with safety to the user, for example, by using a gel forming polymer which is FDA approved, incorporated with low levels of a low-cost, virus capturing polymer and an approved cosmetic ingredient. These two broad-spectrum active components in a liquid gel/cream form are capable of actively interfering with the entry of the viral or bacterial particles into the cells by inhibiting/neutralizing them via molecular interactions. The active ingredients have a synergistic efficacy and moisturizing effects.

The compositions of the technology can provide a passive and/or an active barrier for viral adhesion/binding and bacterial adhesion. Such prophylactic intervention must reduce the viral, respiratory pathogen numbers to sub-infective levels after transmission, with the inhibition of cytopathic effects on human nasal mucosal cells. The attachment to host tissues is a critical step for most pathogens' invasion and dissemination

As an example of a passive mucosal barrier, the mucoadhesive polymer is chosen from a class of mucoadhesive polymers such as hydroxypropyl methylcellulose, carbomers, polyethylene oxide, polyethylene glycol (PEG), polaxamers and the like. These form a passive barrier on the skin or mucosal surfaces. The gel forming polymer functions as a mucoadhesive polymer retaining the formulation contact with the skin and mucosal lining.

Thus, the composition components can include a polymer carrier which may also act as a physical barrier with or without mild surfactant activity

As an example of an active viral microbial barrier, the viral capture/binding polymers belong to the class of multiply charged anionic polymers with sulfonate functionalities and or carboxyl functionalities with molecular weights ranging from about 2000 to about 500,000. A broad-spectrum virus active polymer capable of lowering the virus concentration by binding it and preventing cell attachment can be used.

In the compositions, the antimicrobial or antibacterial component can be chosen from probiotic strains of the Lactobacillus rhamnosus family such as Lactobacillus GG, or L. fermentum, L. Plantarum and the like or heat killed or probiotic derivatives or fermentates. This component can provide a probiotic capable of outnumbering bacterial pathogens and colonization.

The antibacterial antiviral component is also chosen from alpha hydroxy acids and gluconolactone, glyceryl monolaurate and the like, lactic acid and the like.

A synergic action is expected between the sulfonate and the alpha hydroxy acid component creating antiviral and antibacterial due to pH changes or other chelating mechanisms.

Optional lubricants such as glycerin, propylene glycol, Aloe vera can be added, and these optional lubricants are not limited to cosmetic ingredients currently used.

The compositions be provided as lotions, gels, lipsticks, chap sticks, wipes, make up and foundations, deodorant compositions, as well as in disinfecting sprays or surface cleaners.

The final pH for the composition preferably is adjusted to about 5.5-6.

Biopolymers such as basil or chia seed mucilage, alginates, pectin, clay-pectin, clays mineral oxides such as titanium oxide etc. and mixtures can be used as passive barriers. However, these alone will not suffice as a barrier. The idea is to stop and trap the pathogens and also moderate their behavior by an active mechanism, which is described below.

An active viral barrier component is a polyanionic polymer multiply negatively charged poly-sulfonate family of polymers (e.g., polyvinyl sulfonate, polystyrene sulfonate and the like), that prevents/interferes in the attachment or fusion of the viruses with host cell epithelium—as a broad-spectrum agent. An active viral barrier component can function by blocking the interaction of the contacting viral proteins with receptors at the cell surfaces and thus block virus-cell fusion.

Due to the “bind and inactivate” type mechanisms the viral pathogens become unavailable to attach to the host cells due to their capture by the PVS polymer.

An active bacterial barrier component such as lactic acid (EPA registered, FDA approved for direct use in food and cosmetics) as a potent antibacterial as a broad-spectrum agent is active toward gram-positive and gram-negative bacterial pathogens. Due to multiple mechanisms of membrane impairment, oxidative stress, and proton shuttling mechanisms, bacteria can become susceptible to lysis within short exposures to preventative treatment by this component. It also serves as a moisturizing agent.

A possible synergy is anticipated from the unique combination for enhancing the activity toward viral and bacterial species within short contact times about <30 minutes. Charged, sulfonated polymers, with alpha hydroxyl acids, may inherently provide enhanced efficacies as a first line of defense. A synergic action is expected between the sulfonate and the alpha hydroxy acid or amino acid component, creating antiviral and antibacterial due to pH changes or other chelating mechanisms.

These mechanisms are not comparable to virucidal action but rather a barrier with active and passive shielding for about up to a projected 6-8 hrs, under low volume fluid flow condition such as muco-ciliary clearance.

Unlike other areas where the body fluids are complex and flow continuously, the facial skin and nostrils are relatively dry with natural mucus that coats the nasal passages. The compositions herein can add a mucoadhesive coating with a good adhesion to the underlying mucus layer. Lubricating additives such as aloe vera, oils and vitamins can be added.

The present technology provides infection control, for example, in urinary tract infection (UTI), including non-catheter and catheter associated (CA-UTI), which remains one of the most common clinical problems presented to medical practitioners. UTIs are frequently treated with antibiotics, which has exacerbated the emergence of antibiotic resistant strains in the uro-pathogens. Consequently, there is increasing interest in the development of safe alternatives to antibiotics. The use of probiotics has been advocated as a plausible alternative to antibiotics, but questions remain as to their mode of application.

In an example composition, a locally applied, weakly reversible mucoadhesive containing probiotic gel, with daily use will allow continued levels of Lactobacillus at the mucosa surface, blocking the adherence of UTI pathogens and can thus enhance the mucosal barrier properties. Water-soluble, moderately mucoadhesive polymer FDA approved Hydroxy Propyl Methylcellulose (HPMC), can be selected as the swellable carrier for the gel. A customized water dispersible HPMC powder can be used along with a beneficial probiotic Lactobacillus strain with antimicrobial properties (that fights colonization of pathogens). The inhibition of mucosal UTI pathogenic microbial colonization, inflammation reduction, and inhibition of the growth of insoluble calcium, magnesium infection stones are examples of the merits.

UTIs including the recurrent infections account for almost 25-50% percent of all infections in women and 15-40% for men. The prevalence of UTI increases with age in both genders, and women aged over 65 are at a higher risk. The present technology is based on a hypothesis that a steady reduction of the pathogenic bacterial burden in the genitourinary region eventually can reduce the risk of ascension into the urinary tract and the incidence of symptomatic UTI.

Such a single-use, disposable, in situ gel composition is innovative and overcomes issues of probiotic stability, viability, and shelf-life issues. It can be used like any catheter lubricant gel, and the modest mucoadhesive nature allows the retention of the Lactobacillus on the mucosa. This is the first such developmental product. The present design allows mixing without touching the components. The configuration, chemistries, composition, and methodology are each carefully designed. Only specific Lactobacilli strains seem to have the ability to interfere with the adherence, growth, and colonization of uropathogenic bacteria. The compositions herein also exhibit antimicrobial, anti-inflammatory properties, and interfere with the spread of pathogenic organisms within the colonized part. A local delivery approach is more advantageous as it reduces the gastrointestinal exposure and potential side effects of oral probiotics. Stapleton, et al., 2011 report that intravaginal Lactobacillus suppository treatment in human trials decreased UTI and complications significantly compared to the placebo group.

U.S. Pat. No. 9,962,416 claims a method of diagnosing and selecting treatment for a UTI in a subject having a neuropathic bladder (NB), comprising: screening the subject for UTI risk; selecting a proper lower urinary symptom treatment or UTI treatment if the subject's risk of UTI exceeds a threshold, wherein the proper treatment is the administration of a therapeutically effective amount of a probiotic treatment to the subject, wherein the probiotic treatment includes intravesicularly administering to the subject Lactobacillus rhamnosus GG; and administering the proper lower urinary symptom treatment or UTI treatment to the subject.

There are benefits of a prophylactic, instant, user-friendly Lactobacillus gel as a catheter lubricant vs. direct bladder instillations treatment. The gel mechanism involves small amounts of Lactobacillus introduced into the urinary tract (urethra and the bladder) during self-catheterization similar to any catheter lubricating gel. The Lactobacillus cells from the gel populate the mucosal surfaces, potentially conferring a dynamic urethral mucosal barrier for UTI pathogenic adherence as well a reduction in the pathogenic load due to its antimicrobial properties. These include the ability of some Lactobacillus strains to (i) adhere to mucosal surface cells; (ii) exclude or reduce pathogenic adherence; (iii) persist and multiply; (iv) produce acids, hydrogen peroxide, and bacteriocins antagonistic to pathogen growth. Hypromellose (HPMC) is mucoadhesive and increases the retention of the gel after application at the body temperature.

Self-administered direct bladder instillations are used as large single doses, cumbersome, prone to contamination, could demand a high level of commitment, and high compliance on the part of the participant and may not be cost-effective.

The antimicrobial activity of intra-urethrally administered probiotic Lactobacillus casei strain against Escherichia coli in a murine UTI model is successfully demonstrated herein, with a significant reduction in the rate of UTI. Lactobacillus casei, Lactobacillus paracasei, and Lactobacillus rhamnosus form a closely related taxonomic group.

Stapleton, et al. report that intravaginal Lactobacillus suppository treatment decreased UTI and complications significantly, compared to the placebo group in women subjects. Lactobacillus rhamnosus GG (LGG), is a well-studied commercially available strain tested in several animal models that could have the potential to reduce UTI. Lactobacillus crispatus and Lactobacillus plantarum have shown promise.

Benefits of catheter gel over daily bladder instillations: When the lubricant gel, containing Lactobacillus is used during self-catheterization four to five times a day, it is a different approach from the large one or two doses of direct bladder instillations of 20 billion CFU/dose. The gel mechanism involves viable Lactobacillus cells introduced from the gel populating the urinary tract (urethra and the bladder mucosa) continuously at perhaps low levels with the regular use of the presently disclosed special gel. This is only a prophylactic product—unlike a drug, a “therapeutic dose” cannot be defined for the lubricant use as Lactobacillus are live cells that may grow or remain static depending on the conditions in the bladder, but the levels in the single-use gel product can be chosen at the time of use. The microbiome balance can also be slowly restored.

Direct bladder instillations are cumbersome, can demand a high level of commitment and high compliance on the part of the participant, and may not be cost-effective. The instilled solutions become diluted with urine and get washed out of the bladder during voiding. The self-bladder instillation administration process could itself cause contamination via the catheter.

A single-use gel with a similar consistency as the regular catheter lubricating gel can prove user friendly and protect the catheter tip from contamination by UTI pathogens introduced during each self-catheterization.

Local levels of a weakly reversible mucoadhesive containing probiotic gel can allow continuous colonization, outnumbering the pathogens, and could thus enhance the mucosal barrier properties.

Pharmaceutical grade cellulose polymers for enhancing non-covalent interactions with the mucosa with a temporary mucoadhesive property are used extensively in medical products, and such property is expected to enhance the contact time for the probiotic synergistically, and it is non-sticky to touch and washable. The cellulose polymer HPMC may also serve as pre-biotic allowing a favorable environment for the probiotics. The composition components thus can include a medical polymer carrier which may also act as a physical barrier with or without mild surfactant activity

Lactobacillus rhamnosus GG (LGG) ATCC: is a well-studied commercially available strain, tested in several animal models that could have the potential to reduce UTI. Several systematic reviews of small clinical trials and meta-analysis concluded that orally administered probiotic strains of Lactobacillus are safe and effective in preventing recurrent UTI in adult women. Several have been tested in clinical trials, in the urogenital tract in animal models. Auanet and Cochrane reviews have analyzed data derived from several small human studies. It is believed that a local delivery approach is more advantageous as it reduces the GI exposure and potential side effects of oral probiotics, such as diarrhea, nausea, vomiting, and constipation and GU symptoms.

Antimicrobial Mechanisms of Action of Probiotics: A Probiotic component are defined as “a preparation of, or a product containing viable, defined microorganisms in sufficient numbers, which alter the microflora (by implantation or colonization) in a compartment of the host and by that exert beneficial health effects in the host”. Potential mechanisms of probiotic organisms in the genitourinary tract include establishing a barrier against ascension and colonization by reducing uropathogen adherence and growth, as well as affecting immune function. Probiotics such as the Lactobacillus family act by secreting antimicrobial substances such as organic acids, bacteriocins, and hydrogen peroxide. They eventually, by counteract, interfering with the spread of pathogenic organisms within the colonized part, reducing the pH and producing biosurfactants.

Several systematic reviews including a 2004 review on Lactobacillus antagonistic activity and on small clinical trials and meta-analysis concluded that orally administered probiotic strains of Lactobacillus are safe and effective in preventing recurrent UTI in adult women. Several have been tested in clinical trials in the urogenital tract in animal models. Auanet and Cochrane reviews have analyzed data derived from several small human studies.

Oral prophylactics approaches such as methenamine hippurate, d-mannose, cranberry, probiotic supplements, and vitamin C are some approaches that have been clinically tested or on trials. Cranberry and d-mannose treatments have met with limited success.

The instant gel-forming method overcomes Lactobacillus viability issues (by using a commercial dry premix in a package containing the probiotics that is added to sterile premeasured water or buffer provided in a container, followed b the addition of gelling agent as quick dispersing paste) until the consumer chooses to make the gel.

The present technology provides a self-replenishing “live” in situ, lightly mucoadhesive, infection control barrier gel composition with a safe probiotic strain. It has consistencies similar to moisturizers or lubricating gels.

No clumping, tackiness or flakiness was observed and compatibility of HPMC (hydroxyl propyl methylcellulose or hydroxy ethyl cellulose) is excellent. Probiotics was distributed in a highly homogenous smooth, lubricious colloidal gel. Some air bubbles may appear, which should not compromise the performance.

The instant gel once made for a day's supply (for example, 2 or 3 oz) can be supplied in portions, before each catheterization, for example, for intermittent catheter users as a catheter lubricant.

The viability of Lactobacillus in the gel is stable with no significant growth or loss of viability when stored at room temperature even up to 30 days as verified for 72 hours. Therefore, a day's use does not pose an issue.

A range of 0.1 billion cells/g to 100 billion Lactobacillus cells/g of the gel formula could be made.

The gel adherence to mucosal tissue and inhibition of UTI pathogen colonization, and efficacy (bactericidal and bacteriostatic) is anticipated. The gel may be able to impede or reduce inflammation in the bladder due to retrograde bacterial colonization in the case of catheter users.

Lactobacillus is shown to persist in high numbers actively in the presence of intentionally UTI contaminated/co-infected pathogens, and the inhibitory effect of the Lactobacillus on UTI pathogen growth >3 log reduction or 99.9% is demonstrated.

In an embodiment, a mucoadhesive, swellable gel polymer approved for medical use is provided as a paste in a non-aqueous solvent such as glycerin, propylene glycol, polyethylene glycol, and mixtures, along with small amounts of simethicone. This is provided as a specific quantity in a syringe or suitable squeeze tube like foil pouch.

A dry probiotic mix customized from commercial forms with a certified viability and shelf life is provided as an easy to open sachet or pouch.

The user is provided with a disposable bottle containing premeasured phosphate buffered saline or sterile water.

Such a single-use, disposable in situ gel configuration is innovative and overcomes issues of probiotic stability, viability, and shelf-life issues. It can be used like a lubricant gel, and the modest mucoadhesive nature allows the retention of the Lactobacillus on the mucosa.

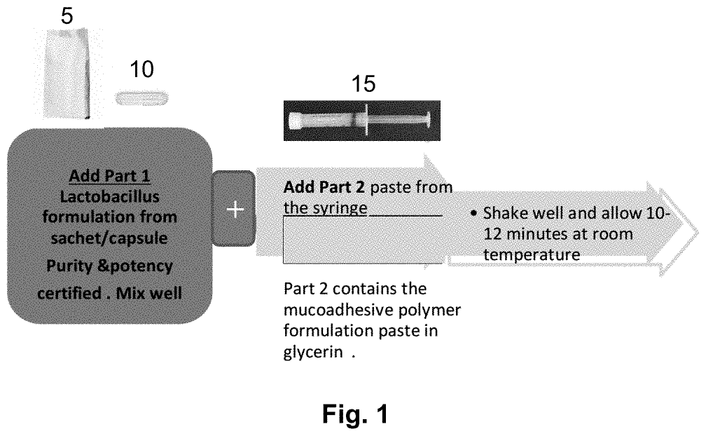

In FIG. 1, an instant gel, single-use configuration is shown. The example of FIG. 1 can be used as a catheter lubricant, as general direct mucosal surface lubricant, or as a topical gel. At left of FIG. 1, Part 1 contains the dry probiotic Lactobacillus formulation powder form, in easy to open notched sachets 5 or capsules 10.

The gel is made within 5-10 minutes at room temperature (about 18-30° C.) by a user-friendly simple mix by shaking for about 30 secs after adding Part 1, followed by the addition of the Part 2 paste, in syringe 15, and usually allowed for about 5 to about 10 minutes, or for about 10 to about 12 minutes, or for about 20 minutes to fully hydrate. The lubricant gel is prepared for each day and used similar to a catheter lubricating gel 5-6 times.

The gel can be made thus as a disposable single package for single use on an as needed basis. The viscosities range from about 5000 cps to about 90,000 cps based on the concentration of the gelling polymer and its molecular weight.

In another embodiment, gel forms are made by quick dissolving capsules that disintegrate upon drawing water or a buffer at room temperature or warmer temperature of about 60° C.

The capsule or pod shell can be made of an FDA approved water soluble polymer.

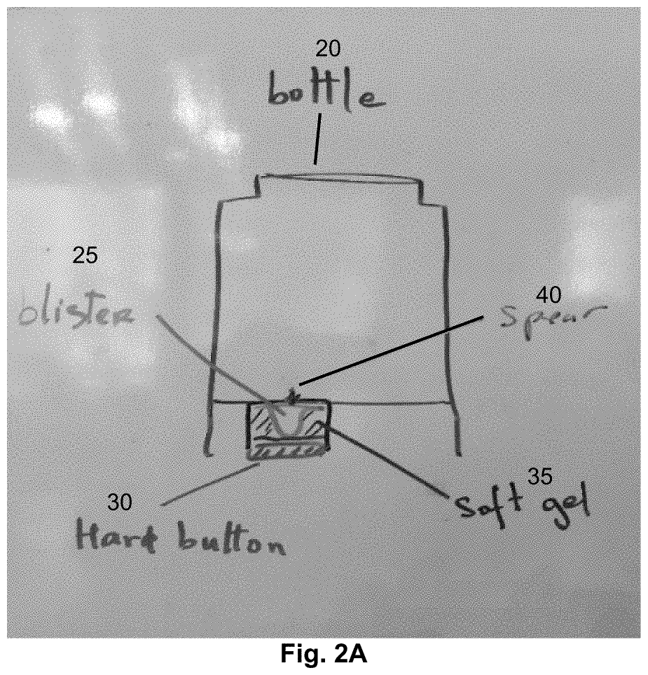

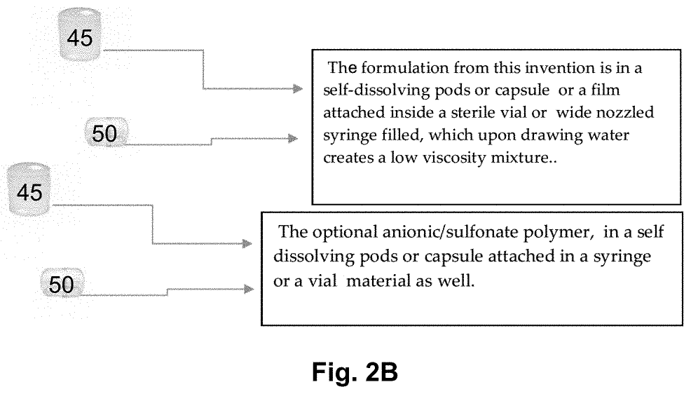

In another embodiment, a gel polymer paste is released from a blister at the bottom of a sterile container to which water and probiotic formulation is added and mixed. FIG. 2A shows an example of a bottle 20 with a blister 25 at the bottom. A button or hard button 30 can be pressed by a user to rupture the spear 40 and release a soft gel 35 into the bottle 20. A small spear 40 can be included in the blister, or blister can be ruptured by pressure without it. Multiple blisters, each with spears, buttons, and contents can be included on one bottle. The blisters can include a ready composition, to which water or other solvent is added. The blisters can include one component or an additional ingredient.

The probiotic component or composition could be kept in another blister pack as well and it would simply require adding water or a buffer. Dry powders of the probiotic compositions can be packaged in sachets, capsules, pods, or other shapes, with zo medically approved dry, swellable hydrophilic polymers (such as cold-water soluble types) and the user is advised to add sterile water or buffer provided at a specific volume and make an “instant gel”. FIG. 2B shows an example of a self-dissolving pod 45 or capsule 50 that can include components, dry powders, or probiotic compositions packaged in medically approved dry, swellable hydrophilic polymers. In FIG. 2B, the pod 45 or the capsule 50 can be in other physical forms, for example, sachets, beads, patches, and multiple compartments, self-dissolving configurations. The pod 45 or capsule 50 can be attached inside a sterile vial, bottle, or wide nozzle syringe. For example, when the syringe is filled, a mixture or low-viscosity mixture is created inside the syringe. In another embodiment, the antimicrobial component is blended with the swellable polymer in one package and the polyanionic polymer such as polyvinylsulfonate is present in the water. The optional anionic/sulfonate polymer can be provided in pod 45 or capsule 50. The user is advised to drop the pods 45, capsules 50, or sachets in a sterile vial, add sterile water provided, shake and use after 30 minutes. The biocompatible sachet or pod material itself contributes to the gel viscosity and blends with the formulation to provide a homogenous viscous gel like consistency. The example configurations shown in FIG. 2B can be combined with the example of FIG. 2A.

Yet another embodiment is a dry protected water-soluble formulation in the form of a patch applied that becomes a biocompatible hydrogel on contact with water during use or could be wetted in sterile water before use. For example, the insertion of urinary catheters can be done via the hydrated patch and allowing the patch to remain at the catheter/urethra site to inhibit catheter associated infections into the bladder.

In another application, such disposable dry patches can be instantly used by adults for urinary tract infection/vaginal infection control via an adhesive strip on the underwear.

In another embodiment, a highly swellable sponge, diaper, or textiles impregnated with dry compositions are wetted and applied on surfaces to prevent pathogen transmission.

Non-aqueous polymeric vehicles with sufficient viscosity such as liquid silicone polymers, (medical grade silicone fluids, simethicones), squalene types, polyethylene glycols, lipid or oil based and other approved hydrophobic carriers, and petroleum-based vehicles may be used,

In yet another embodiment the compositions could be incorporated in dry polymer films especially with the probiotic or environmentally sensitive components and dissolved in situ to create a gel.

In another embodiment, the components can be configured in a 3D printed design or print pattern.

As used herein, the term “mucoadhesive” denotes a composition having a reversible adhesive property with various degrees of mucoadhesion.

### EXAMPLES

In the disclosures herein, examples with specific strains in commercially available products for the purposes of demonstration and the scope is not limited by the probiotic strains, gel forming polymer, prebiotic additives.

If probiotic components are used the cell viability and probiotic survival in the final products are important that they are preserved.

Potentiators and antioxidants can be included as additional in any of the compositions, methods, and configured herein. The addition of small amounts of sugars such as sucrose, to the formulation could be beneficial to boost the growth of Lactobacillus so it is able to outrun the growth of pathogenic organisms. Similarly, the addition of ascorbate may have a potentiating effect along with inherent antibacterial activity of ascorbic acid which is produced over time when ascorbate is added. Lactate/lactic acid could also prove useful.

Lactobacillus rhamnosus GG (LGG) CULTURELLE products were chosen for the sake of demonstration, as LGG potency is certified in such commercial products with shelf life information under proper storage conditions. There is also literature support on its safety (Lactobacillus casei, Lactobacillus paracasei, and Lactobacillus rhamnosus form a closely related taxonomic group).

However, this would not restrict the technology from using other Lactobacillus sources or strains or combinations of strains. Examples are Lactobacillus crispatus, Lactobacillus fermentum or Lactobacillus plantarum and the like.

Similarly, the gel forming polymers could be chosen from compatible cellulose based vegan types, animal-based gelatin powder, or plant-based mucilage types which as polysaccharides. Cellulose polymers—polysaccharide combinations are also possible.

### Example 1. Composition Ingredients and General Format

Compositions were prepared to demonstrate the addition of the components described above to form a clear gel or a white gel. Table 1 presents example compositions of both. In this example, the same format could be used in sterile water instead of PBS.

TABLE 1A
Example compositions and general format.

| Composition | Notes |
|---|---|
| Hydroxyl propyl methylcellulose (HPMC) in phosphate | clear gel |
| buffered saline solution (PBS) | |
| Polyvinylsulfonate (PVS) (soluble anionic polymers) + | clear gel |
| HPMC + Phosphate buffered saline solution. | |
| Lactobacillus Rhamnosus GG ( CULTURELLE™⁻brand) + | white gel |
| HPMC - No PVS + phosphate buffered saline solution. | |
| Lactobacillus Rhamnosus Lr 32™ (CUSTOM | white gel |
| PROBIOTICS), | |
| as supplied in their commercial form, with PVS + HPMC + | |
| phosphate buffered saline solution. | |
| Lactobacillus Rhamnosus GG (CULTURELLE™) as | white gel |
| supplied in their commercial form+HPMC + PVS | |
| +phosphate buffered saline solution. | |
| Lactobacillus Rhamnosus Lr 32™ (Custom Probiotics.com) | white gel |
| as supplied in their commercial form - No PVS + HPMC | |
| +phosphate buffered saline solution. | |
| LifeinU*™ Lactobacillus rhamnosus GG as supplied in | white gel |
| capsules. | |

*LifeinU™ Lactobacillus rhamnosus GG, Lesaffre, France. Sold by
us.supersmart.

### Example 2. Preparation of Instant and Two-Part Gels

Part 1: is a sterile aluminum foil pouch or a sachet containing the required amount of dry probiotics as supplied and the pouches are heat sealed. This could also be in the form of capsules, pods, or other forms with the required amounts filled. For example, it could be customized to contain sufficient potency of the Lactobacillus colony forming units along with prebiotics such as Inulin (water soluble) or cellulose polymers that are water soluble. Other antioxidants/potentiators such as ascorbate or lactate may be added.

Preparation of the Part 2 paste containing Hydroxypropyl methylcellulose in Glycerin: Dry 14.402 grams of Hydroxypropyl methylcellulose HY1136 is ground in a blender with 3.6 grams of ultrapure Phosphate buffered saline powder 10× concentration. This is slowly added to warm (˜70° C.) 60 grams of Glycerin with vigorous stirring. The paste is stored in sterile jars. The pastes are filed in medical grade syringes at the required quantities and sterilized by steam after packaging.

TABLE 2
The HPMC paste composition (Part 2)

| Ingredient | Source | Weight percent % |
|---|---|---|
| Hydroxypropyl | USP grade; spectrum | 18.4% |
| methylcellulose | chemical Hypromellose | |
| USP grade | grades 1136 | |
| PBS 10X powder | Spectrum chemical, P3027 | 4.5% |
| ultrapure, USP | | |
| grade | | |
| Glycerin, USP | Spectrum chemical | 76.7% |
| Simethicone, USP | Spectrum chemical | 0.3% |
| grade | | |

General Method of making the gel: The contents of the CULTURELLE capsules (200-350 mg) and contents of the sachets (˜1-1.5 gm) were transferred weighed into an aluminum foil fin sealed pouch under aseptic conditions and heat sealed. In this example two Children's purely probiotic sachets (5 billion CFUs/sachet) were mixed with two capsule contents of Ultimate strength capsules (20 billion cfu/capsule). It was dispersed in sterile PBS or water and the PBS added with the HPMC paste. The compositions were uniform and had a good consistency of with viscosities of the order of 20,000-25,000 cps at 20° C. Viscosities could be adjusted to a range from 10-80,000 cP.

The Cutlurelle™ capsule contents that had Inulin had a better dispersion than the capsule content that the one which had microcrystalline cellulose, though this is not a critical issue. Inulin is pre-biotic that might help boost the growth of the Lactobacillus in the presence of pathogenic.

Table 1B shows examples of optional sodium ascorbate and optional sugars.

TABLE 1B
Table of sodium ascorbate and sugars.

| Ingredient | Source | Weight % |
|---|---|---|
| Optional Sodium | Spectrum | 0-2% |
| ascorbate, | chemical | |
| Sugars, such as | | 0.01-0.5% |
| Sucrose | | |

Tables 2A, 2B1, and 2B.2 show two-part gel formulations. Table 2A shows a gel formulation (coded as IC-2A) with a Lactobacillus fermentate provided as illustrated in the table. The final mixture in PBS or distilled water is well vortexed at stored at room temperature.

Table 2B.1 shows a formulation example with a Lactobacillus rhamnosus Lr32 (coded as IC-2B1) probiotic provided as illustrated in the table.

Table 2B.2 shows a formulation example with a Lactobacillus rhamnosus GG probiotic (coded as IC-2B2) provided as illustrated.

TABLE 2A
Gel formulation IC-2A

| Ingredient | Source | Weight percent |
|---|---|---|
| Part 2 | USP grade; spectrum | 1-2% |
| Hydroxypropyl | chemical | |
| methylcellulose | Hypromellose | |
| paste in Glycerin: | grades 1136 | |
| PBS solution | Spectrum chemical | 87-98% |
| ultrapure | | |
| Part 1. LEUCIDAL | Lotion crafter, WA | 1-11% |
| SF max™ | | |

TABLE 2B.1.
Formulation example IC-2B1

| Ingredient | Source | Weight |
|---|---|---|
| Part 2. Hydroxypropyl | Spectrum | 1.2-3% |
| methylcellulose 1136 paste in | chemical | |
| Glycerin | | |
| Part 1. Lactobacillus | Probiotics.com | 1-15% |
| Rhamnosus Lr32 powder, | | |
| potency labeled as 1g =200 | | |
| billion cells. | | |
| Sterile Phosphate buffered | Spectrum | 82-97.8% |
| saline solution | chemical | |

TABLE 2B.2.
Formulation example IC-2B2

| Ingredient | Source | Weight percent |
|---|---|---|
| Part 2 Hydroxypropyl | Spectrum chemical | 1-3% |
| methylcellulose paste in | | |
| Glycerin: | | |
| Part 1 Lactobacillus | brand CULTURELLE™ | 1- 20% |
| Rhamnosus GG (LGG) | | |
| various commercial | | |
| capsules and or sachets | | |
| or combinations of both | | |
| with certified potencies. | | |
| Potency of 10-20 | | |
| billion/capsule and 5 | | |
| billion cfu /sachet. | | |
| Or stabilized LGG powder | | |
| Sterile Phosphate | Spectrum chemical | 66-96% |
| buffered saline solution or | | |
| sterile water | | |

An example of a non-aqueous, instant version is shown in Table 2C below.

TABLE 2C.
Example of non-aqueous instant version.

| Ingredient | Source | Weight percent |
|---|---|---|
| Simethicone | Spectrum chemical | 0-9% |
| Natural oils | Spectrum chemical | 0-1 % |
| Lactobacillus Rhamnosus GG | brand CULTURELLE^{M} | 1- 50% |
| commercial capsules and or | | |
| sachets or combinations of | | |
| both with certified potencies. | | |
| Potency of 10-20 | | |
| billion/capsule and 5 billion | | |
| cfu /sachet. | | |
| MDM silicone oil | | 49-99% |

Table 2D demonstrates the use of an existing water-based lubricating gel to create the instant use gel. The probitotic powder is dispersed in water at room temperature and the commercial gel is added and shaken well.

TABLE 2D
Instant gel using an existing lubricating gel or ointments.

| Ingredient | Source | Weight percent |
|---|---|---|
| Carbomers, Hydroxy ethyl cellulose, | Lubricating jelly / | 50-80% |
| Hydroxymethyl propyl cellulose, | gel like HR | |
| glycerin, propylene glycol | lubricating, E-Z, | |
| | and Mckesson | |
| | etc. as sold wth | |
| | viscosities | |
| | ranging from | |
| | 20cps- 60,000 | |
| | cps. | |
| Lactobacillus Rhamnosus GG | brand | 5-20% |
| commercial capsules and or sachets | CULTURELLE^{M} | |
| or combinations of both with certified | | |
| potencies. Potency of 10-20 | | |
| billion/capsule and 5 billion cfu | | |
| /sachet. | | |
| Water | | 15-30% |

The method lowers the viscosity of the resulting gel dramatically and use of saline has a dramatic effect in lowering viscosities in gels like Mckesson. In addition, the viscosity adjustment and uniformity of the probiotic dispersion could be overcome by those skilled in the art.

### Example 3. Biocompatibility Assays

Biocompatibility assays were performed by PerfectUs Biomed group (formerly Extherid) on the samples of IC-2B2 samples from Example 2, Table 2B.2

Results are shown in Table 3 below. MTT assays returns relative viability of tissue through calorimetric analysis. Results are reported as percent viability compared to the non-irritative PBS control, which represents 100% viability for each of the four test conditions.

Mild and moderate irritant control compounds TX-100 and SAS, respectively, demonstrated an expected decreases in tissue viability for all test conditions. Larger quantities of irritant result in lower viability, as seen in 100 μL trials. Compounds do not show significant differences in viability between 2 h and 24 h timepoints, which indicates any cytotoxic effect is induced in less than 2 h.

Both UT20 23 formulations (Table 3) demonstrated excellent biocompatibility after 2 h and 24 h timepoints when applied at a 10 μL dose, evidenced by equivalent colorimetric data to the PBS control. UT20 formulations demonstrate greater than 100% viability in certain trials. Because 100% viability for each trial is represented by the colorimetric average of the 3 explants treated with PBS. viability greater than 100% indicates UT20 formulations impart cytoprotective effects compared to the neutral PBS control.

In Table 3 below, gel formula numbered UT20 23-01 is made of HPMC and CULTURELLE™ capsule contents (Digestive balance—its ingredients listed on the carton; contains Inulin) at a concentration of approximately one billion cfu/gm of the formula. Gel formula (IC-2B2 type) numbered UT20 23-02 is made as above with approximately 2 (two) billion cfu/gm of the gel. In Table 3, zone of inhibition (ZOI) measurement is measurement of inhibition diameter in millimeters.

TABLE 3
Zone of inhibition (ZOI) measurements

| ICET ID | K. pneumoniae | E. coli | C. albicans | P. mirabilis | E. faecalis |
|---|---|---|---|---|---|
| UT20 23-01 | 31 | 29 | 20 | 35 | 0 |
| UT20 23-02 | 26 | 39 | 41 | 29 | 0 |

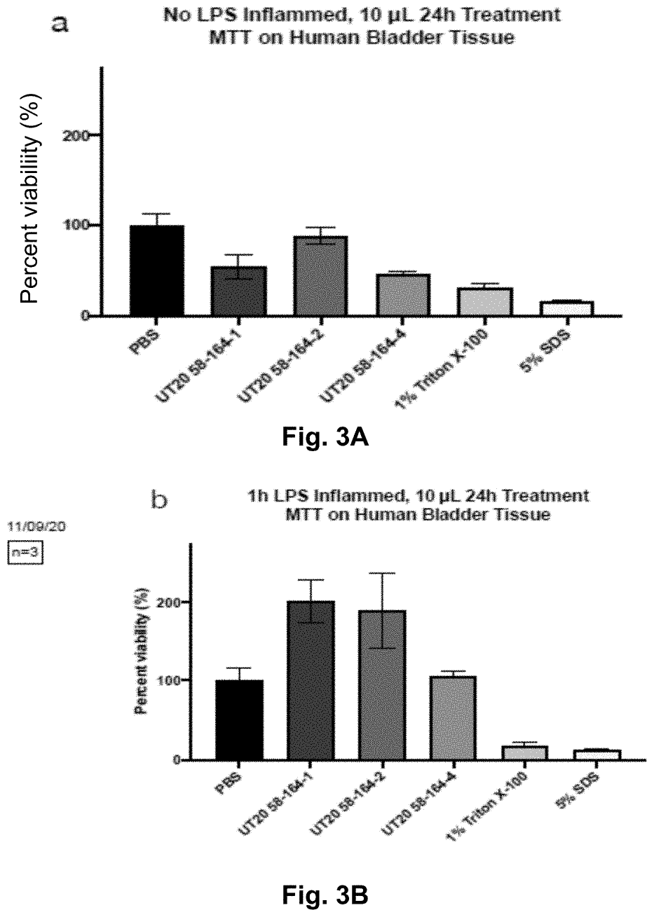

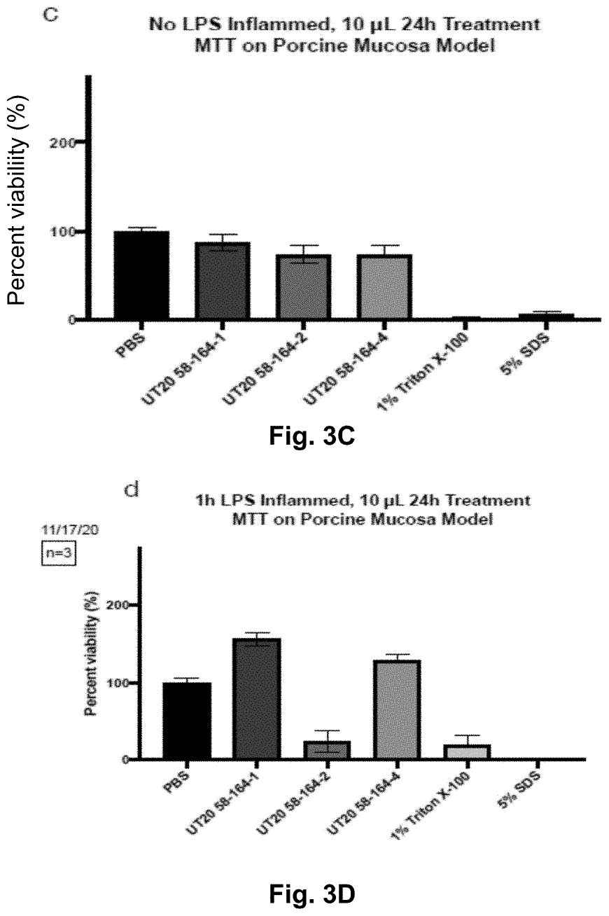

FIGS. 3A-3D show results of the UT20 58 series MTT biocompatibility assay. In FIGS. 3A-3D, IC2B2 samples labeled 164-01: Made with HPMC and two capsules containing LGG and microcrystalline cellulose. LGG concentration approximately 1.3 billioncfu/mL; IC2B2 sample labeled 164-02: When made with CULTURELLE's Purely probiotic children's sachet (5 billion cfu/ sachet); there was a zone of inhibition with most UTI organisms including Enterococcus faecalis. LGG approximately 0.66 billion cfu/gm of the gel.

FIGS. 3A shows MTT assay assessing viability of human bladder tissue explants after 24 h treatment with 10 μL doses of ICET UT20 58 product compositions. Active compositions are compared against a commercial control (UT20 58-164-4), a non-irritative control (PBS, phosphate buffered saline), a mild irritant control (1% TX-100, Triton X-100 surfactant). FIG. 3B shows same experimental design as FIG. 3A except tissue explants are pre-treated with 10 μL lipopolysaccharide (LPS) for 1 h. FIG. 3C shows same experimental design as FIG. 3A except human bladder tissue was replaced with porcine vaginal tissue. FIG. 3D shows same experimental design as FIG. 3B except human bladder tissue was replaced with porcine vaginal tissue.

The addition of LPS resulted in reactive damage to the bladder sections with a loss of cells on the surface of the section. The addition of LPS with TX100 resulted in mild cell loss and submucosal edema. Together these results illustrate the limited range of reactivity of the human bladder model. By comparison, the porcine vaginal mucosa lost normal structure entirely when LPS was combined with TX100, implying a limit of irritation that can be applied to the model.

The histology reflects a combination of the limits of each tissue models in addition to the reaction to variously added agents. SDS is clearly toxic to the tissue models and the addition of TX100 to the porcine vaginal mucosa removed the ability to assess unambiguously the effect of LPS with a surface active agent such as TX100. The results observed with the addition of agents 164-1, -2, and -4 suggest that either they are protective against the influence of LPS or the degree of insult caused by the LPS was insufficient to determine any protective behavior using histology alone. Table 4 shows results of preliminary histology scores on ex-vivo human bladder and porcine bladder.

TABLE 4
Results of preliminary histology scores.

| | | Qualitative | | Qualitative |
|---|---|---|---|---|
| | HBT | Score | PMM | Score |
| -LPS 10uL PBS | M20 67 | 1+ | M20 79 | 0 |
| -LPS 10uL 164-1 | M20 68 | 1+ | M20 80 | 0+ |
| -LPS 10uL 164-2 | M20 69 | 1+ | M20 81 | 0+ |
| -LPS 10uL 164-4 | M20 70 | 2+ | M20 82 | 0+ |
| -LPS 10uL TX100 | M20 71 | 1+ | M20 83 | 2+ |
| -LPS 10uL SDS | M20 71 | 4+ | M20 84 | 4+ |
| +LPS 10uL PBS | M20 72 | 2+ | M20 85 | 0+ |
| +LPS 10uL 164-1 | M20 74 | 2+ | M20 86 | 1+ |
| +LPS 10uL 164-2 | M20 75 | 2+ | M20 87 | 0+ |
| +LPS 10uL 164-4 | M20 76 | 2+ | M20 88 | 0+ |
| +LPS 10uL TX100 | M20 77 | 3+ | M20 89 | 3+ |
| +LPS 10uL SDS | M20 78 | 4+ | M20 90 | 4+ |

Overall, the Lactobacillus rhamnosus concentrations from 0.1 billion/gm of the final gel to 200 billion/gm of the gel could be made. The preferred concentration for biocompatibility is predicted to be 0.1-50 billion/gm of the final gel.

Versions as ointments, coatings, pellets, inserts, tampons coatings, sanitary napkins, adult or baby diapers or creams could be prepared.

### Example 4. UTI Pathogens

In this example, growth and viability from IC-2B2 Lactobacillus gels in synthetic and human urine medium when contaminated with UTI pathogens are studied. IC-2B2 gel formula (2 ml) with 104 CFU/ml of each culture are used.

Inoculated organisms were individually C. Albicans (ATCC 90028), E. coli (ATCC 700928), E. faecalis (ATCC 51299), K. pneumoniae (ATCC 700603), and P. mirabilis (ATCC 2924). Results are summarized in Table 5.

TABLE 5
IC-2B2 with UTI pathogens.

| Pathogens & | Lactobacillus | Lactobacillus |
|---|---|---|
| Formulation | CFU/ml | viability |
| E. coli & IC-2B2 | 1.2 x 10⁹ | Lactobacillus growth |
| E. Faecalis & IC-2B2 | 2.8 x 10⁹ | Lactobacillus growth |
| K. pneumoniae & IC-2B2 | 3.0 x 10⁸ | Lactobacillus growth |
| P. Mirabilis & IC-2B2 | 2.0 x 10⁸ | Lactobacillus growth |
| C. Albicans & IC-2B2 | 5.1 x 10⁹ | Lactobacillus growth |

### Example 5. Zone of Inhibition Against Uropathogens

TABLE 6
Results of Agar Spot on Lawn Assay (IC-2B1).

| | ZOI (mm) | |
|---|---|---|
| Bacteria | IC-2B1 | Control non-probiotic |
| E. coli | 0.8(0.4)/0.8(0.4) | n.a/ 0.0 |
| P. mirabilis | 0.8(0.3)/0.7(0.3) | n.a/ -0.1 |
| K. pneumoniae | 0.8(0.4)Z 0.9(0.45) | n.a/ 0.0 |

TABLE 7
Agar spot on lawn method against four
bacterial and one fungal strains (IC-2B2).

| Microbes | IC-2B20) |
|---|---|
| E. coli 700928 | 1.5 (0.6) |
| E. faecalis 51299 | 1.4 (0.6) |
| K. pneumoniae 700603 | 1.6 (0.8) |
| P. mirabilis 2924 | 1.2 (0.4) |
| C. albicans 90028 | 0.5 (0.1) |

(1) Parenthesis indicates ZOI from the outside of the formulation.
2) A 100 pl of 104 CFU/ml was plated, and 50 pl was spotted

### Example 6. Contaminated Catheter Tip Models

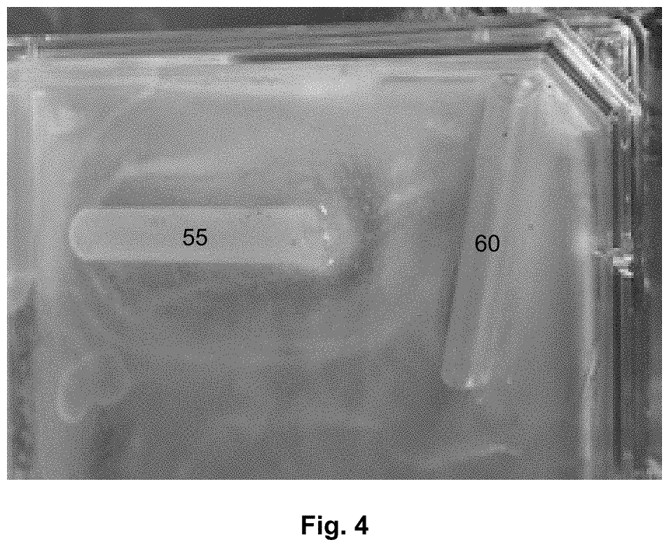

FIG. 4 shows a photo of catheter tips with IC-2B1-type gel placed on E. coli (ATCC 700928 in Table 8 below); the inoculated plate (55, left) shows a zone of a clearing where the gel was applied in contrast to the control commercial gel (60, right), which shows no clearing. Evidence of UTI microbial inhibition by the applied gel composition on catheter tips is further demonstrated by Table 8 below.

TABLE 8
Inhibition of various microbes (catheter tip, IC-2B1 gel)

| Contaminating | ZOI (mm) around the catheter tip |
|---|---|
| Microbes | IC-2B1 gel type |
| E. coli 700928 | 15 |
| E. faecaiis 51299 | 14 |
| K. pneumoniae 700603 | 16 |
| P. mirabilis 2924 | 12 |
| C. Albicans 90028 | 6 |

### Example 7. Calcium Crystal Models

The data in Table 9 below demonstrates the Inhibition of Proteus Mirabilis, resistance to pH Increase and crystal formation. We have assessed the >95% efficacy of the gel treatment on contaminated catheter tips with a urease producing P. mirabilis strain with appropriate controls. P. Mirabilis can produce high levels of urease, which hydrolyzes urea to ammonia (NH3) and makes the urine more alkaline, which can result in bladder stones.

This is significant as the increase in pH with calcium Ca2+ and magnesium Mg2+ cations present in the urine lead to the crystallization, at varying levels of carbonate apatite (Ca10(PO4)6CO3; CA) struvite (magnesium ammonium phosphate hexahydrate; MgNH4PO4.6H2O) and calcium mono oxalate. Struvite (MgNH4PO4.6H2O) and carbonate apatite (Ca10 (PO4)6CO3; CA) are the significant components of recurring infectious urinary stones, which is a painful condition. PVS is a polymeric inhibitor for crystal formation; if present it influences the rate of crystal formation.

Lactic acid produced by the Lactobacillus is also a good chelating agent which produces soluble complex calcium lactate by reacting with calcium. Calcium lactate a soluble complex of calcium can easily be removed from the body, just like citrate complex of calcium. This chelating property may itself control bladder stones.

Polyvinyl sulfonate in addition to mucosal barrier enhancing property also inhibits crystal nucleation and delays growth of crystals by modifying its surface. It is a polymeric inhibitor.

TABLE 9
P. Mirabilis inhibition by interference of growth
by the lactobacillus in the gel preventing the increase in
pH which in turn cause insoluble calcium crystals
(cloudiness) in urine media.

| Gel | | | pH after 24hr | Observations |
|---|---|---|---|---|
| Formulation | | | incubation | of the |
| with live | | | with P. | contaminated |
| lactobacillus | | | Mirabilis (PM) | catheter dipped |
| (LB). | Medium | Initial pH | overnight/plating | solution |
| IC-2B2 with | Human | ~7 | 4-5 significant | No ammonia |
| PVS at 1 % | urine | | Lactobacilli | odor,clear |
| | | | colonies; no | |
| | | | evidence of PM | |
| | | | colonies seen | |
| IC-2B1 | Synthetic | ~5.5 ~6 | 5” | No ammonia |
| | urine | | | odor,clear |
| IC-2B1 | Human | ~7 | 5” | No ammonia |
| | urine | | | odor,clear |
| IC-2B2 | Synthetic | ~5.5 ~6 | 4-5” | No ammonia |
| | urine | | | odor,clear |
| IC-2B2 | Human | ~7 | 6” | No ammonia |
| | urine | | | odor,clear |
| IC-2B2 | Synthetic | ~5.5 ~6 | 4-5 ” | No ammonia |
| | urine | | | odor,clear |
| None, control | Human | ~7 | ~9; Swarming P. | Cloudy, strong |
| | urine | | Mirabilis | ammonia odor, |
| | | | colonies | white crystals |
| None, control | Synthetic | ~5.5 ~6 | ~9; Swarming P. | Cloudy, strong |
| | urine | | Mirabilis | ammonia odor and |
| | | | colonies | white crystals |

### Example 8. The Importance of Kinetic Inhibition

A period of time elapsing between the achievement of super saturation and the observation of the appearance of a new solid phase (i.e. nuclei) or crystals which are often referred to as the induction or the initiation period and is often a sensitive measure of the effectiveness of an additive in inhibiting crystallization. We characterized the kinetic inhibitor properties, because they are evident at much lower inhibitor concentrations than required for equilibrium effects and allowed the comparison of inhibitors.

Effect of the probiotic fermentate Leucidal™, the IC-2A component from example 2A, on Urease action on Urea and crystal formation:

5-11% (w/w) Leucidol was incubated with a 0.1 mL aliquot of 20 unit/mL urease solution (prepared from 40150 units/g urease powder) in SU medium at 37° C. At 60 minutes, the solution had an 86±4.8% reduction. At 150 minutes, the reduction decreased slightly to 74±2.9%.

The crystallization process in urine occurs due to the splitting of urea by Urease producing organisms such as P. Mirabilis forming ammonia, which increases the alkalinity and precipitation of insoluble salts. Crystal formation was determined by observation of turbidity by UV at 660 nm. After incubation overnight in urine media. Mineral components in the artificial or synthetic urine correspond to mean concentration found in 24 hours in normal human urine.

### Example 9. Nasal/Facial Gel Compositions

This example illustrates a nasal/facial gel general composition (NS A and B) composition that could be premade and shelf stable at least up to 4 months. NS A does not contain polyvinyl sulfonate while NS B contains polyvinyl sulfonate.

TABLE 10
Nasal/facial gel composition

| Ingredient | Source | |
|---|---|---|
| Hydroxypropyl | Spectrum | 1.5-2% |
| methylcellulose | chemical | |
| HY124 | | |
| Lactic acid | Sigma | 0.2-0.4% |
| adjusted to pH=6 | | |
| Polyvinyl sulfonate | Sigma, | 0.5-2% |
| Ave.Mw 2000; | Polysciences | |
| 7000 | | |
| Sold as a 25- | | |
| 35w/w % solution) | | |
| Glyceryl | Monolaurin | 0.01-0.3% |
| monolaurate | | |
| F127 polaxamer | Spectrum | 0.05-0.2% |
| Glycerin | spectrum | 0.2-0.7 |
| Polyethylene | Spectrum | 0.1-0.5% |
| glycol 400 | | |
| pH =6 buffer in | Spectrum | Rest |
| water | chemical | |

These compositions are prepared using a buffer of pH=6 with or without pH adjusted lactic acid, that is similar to the nasal pH, followed by the addition of PVS (25% solution) and Polaxamer in water and adding the HY124 or in glycerin as a paste with glycerin as in example 2. No addition of PBS powder is required. Glyceryl monolaurate being insoluble in water is pre-dissolved in PEG 400 or glycerin at about 70° C. The HY124 is slowly added with stirring to form a homogeneous gel of viscosities of about 3000-6000 cps.

ZOI results for both compositions are compared with control in Table 11 below. ZOI is measurement of inhibition diameter in mm.

TABLE 11
The ZOI produced measurements.

| | S. aureus | K. |
|---|---|---|
| | Xen30 | pneumoniae |
| NS control | 0 | 0 |
| NS A(166A) | 25 | 3 |
| NS B (166B) | 25 | 7 |

Further testing was conducted against respiratory pathogens, and results are shown in Table 12 (ZOI in mm) below.

TABLE 12
Effect of lactobacillus instant gel compositions against respiratory pathogens

| | S. aureus | K. pneumoniae ATCC | S. pneumoniae ATCC |
|---|---|---|---|
| | Xen30 | 700603 | 49619 |
| IC-2B2 164-01 | 18 diffuse | 16 diffuse edges | 25 diffuse edges |
| | edges | | |
| IC-2B2 164-02 | 20 distinct | 17 distinct | 31 distinct |
| | edges | | |

### Example 11. Biocompatibility MTT Assays, Histology and Reduction of Inflammatory Biomarkers Upon the Application of the iCET Formulations

A panel of controls for preliminary model development was assessed for biocompatibility properties on Porcine nasal tissue via MTT, and ELISA Assays.

This ELISA assay measures the concentration of the pro-inflammatory cytokines Interleukin 8 (IL-8) and Interleukin 1 alpha (IL1α).

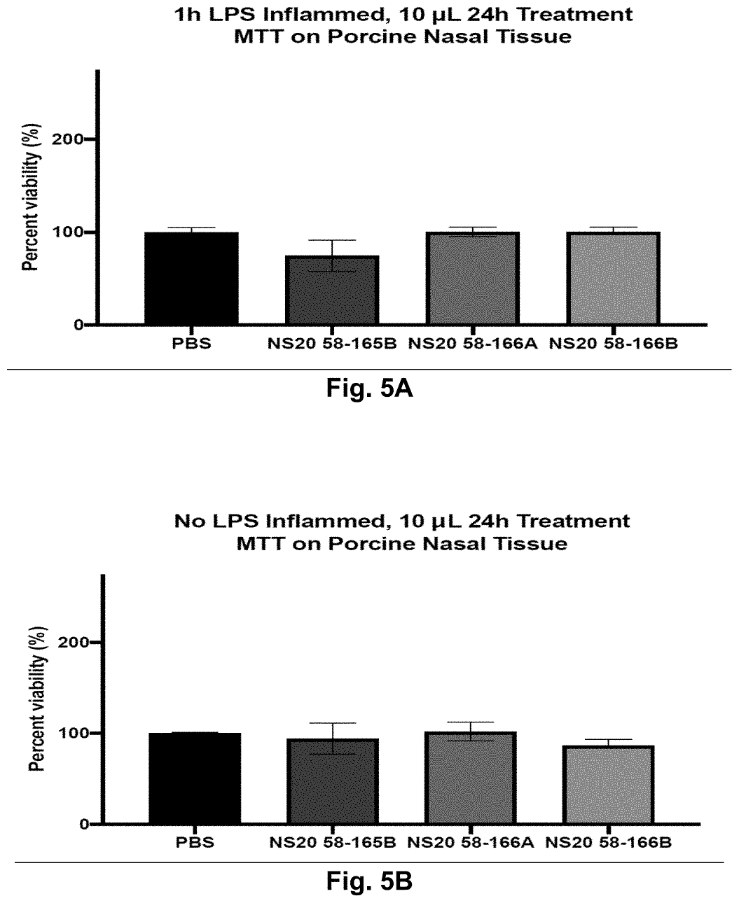

FIG. 5A shows a biocompatibility MTT assay, histology, and reduction of inflammatory biomarkers upon the application of the iCET compositions, of 1 h LPS inflamed, 10 μL 24 h treatment MTT on porcine nasal tissue. FIG. 5B shows the biocompatibility MTT assay, histology, and reduction of inflammatory biomarkers upon the application of the iCET compositions, of no LPS inflamed, 10 μL 24 h treatment MTT on porcine nasal tissue.

### Example 12. Viral Attachment Inhibition to MDCK Cell Lines: Virucidal/Virustatic Properties

Study 1 Protocol Summary: 100 μl of ICET Test samples were deposited at the bottom of glass tissue culture plate. The composition of NS A and NS B is described in Example 9.

TABLE 13
Human Coronavirus.

| | | Log | % |
|---|---|---|---|
| Sample | Virus | Reduction | Reduction |
| ICET NS A | Human Coronavirus OC-43 | 1.44 | 96.40 |
| ICET NS A | Human Coronavirus OC-43 | 1.94 | 98.86 |
| ICET NS B | Human Coronavirus OC-43 | 1.81 | 98.47 |
| ICET NS B | Human Coronavirus OC-43 | 1.81 | 98.47 |

The 2 h control showed killing by the virus.

The above study was repeated with Influenza H1N1 (see below). 20 ul of Influenza A H1N1 virus (2.8×10⁷) was added and incubated for 2 h exposure at RT. The reactions were stopped and serial dilutions made for either cytopathic effect (CPE) or plaque reduction assay.

TABLE 14
Human Influenza.

| Sample | Virus | Log Reduction | % Reduction |
|---|---|---|---|
| ICET NS A | Human Influenza A H1N1 | 2.85 | 99.86 |
| ICETNS B | Human Influenza A H1N1 | 0.24 | 42.86 |

Study 2 protocol summary: Cells were treated with 100 μl of 1:10 ICET Gel dilution for 2 h at 37° C. and then challenged for 1 h at 35° C. with Human Coronavirus OC43 (11 ul of 2.14×10⁷).

TABLE 15
Coronavirus OC-43.

| | | Log | % |
|---|---|---|---|
| Sample | Virus | Reduction | Reduction |
| ICET NS A | Coronavirus OC-43 | 0.75 | 82.22 |
| ICET NS A | Coronavirus OC-43 | 1.50 | 96.84 |
| ICET NS B | Coronavirus OC-43 | 1.75 | 98.22 |
| ICET NS B | Coronavirus OC-43 | 1.00 | 90.00 |

Initial testing of ICET compositions demonstrated antiviral and antibacterial properties against respiratory pathogens. The ICET compositions did not show any cytotoxicity effect at 1:3 dilution.

TABLE 16
Scoring

| | | | Ordinal | |
|---|---|---|---|---|
| Porcine Nasal Tissue | tube # | block # | score | Label |
| +LPS 10uL PBS | 1 | M20 91 | 4 | L2091 |
| +LPS 10uL 165B | 2 | M20 92 | 3 | L2092 |
| +LPS 10uL166A | 3 | M20 93 | 1 | L2093 |
| +LPS 10uL 166B | 4 | M20 94 | 4 | L2094 |
| -LPS 10uL PBS | 5 | M20 95 | 2 | L2096 |
| -LPS 10uL 1668 | 6 | M20 96 | 0 | L2096 |
| -LPS 10uL 166A | 7 | M20 97 | 2 | L2097 |
| -LPS 10uL 1668 | 8 | M20 88 | 0 | L2098 |
| LPS | 9 | M20 99 | 1 | L2099 |
| SDS | 10 | M20 100 | 4 | L2100 |
| TX100 | 11 | M20 101 | 3 | L2101 |

In Table 16, histology scoring of NS A (166A) and NS B (166B) type compositions on porcine nasal mucosa. Sample 165B is a commercial control. It is of some interest that the treatments appear to result in a reduced response with or without the addition of LPS. The detergents SDS and TX100 produce a maximal response while the LPS produces a range of responses. The favorable biocompatibility and anti-inflammatory properties of ICET NS A and ICET NS B products corroborating the results indicate they are suitable to continue testing for commercial application. Both compositions also possessing non-inflammatory properties based on low levels of secreted IL1α. Because IL1α ELISA data mirrors the MTT viability results, it is suggested this panel be used to inform a decision. The blinded participation of the Histology expert lends additional strength to the data.

### REFERENCES

¹ Toh S L, Lee B B, Ryan S, et al. Probiotics [LGG-BB12 or RC14-GR1] versus placebo as prophylaxis for urinary tract infection in persons with spinal cord injury [ProSCIUTTU]: a randomized controlled trial. Spinal Cord. 2019;57(7):550-561. doi:10.1038/s41393-019-0251-y
² van de Wijgert, JHHM, Verwijs, M C. Lactobacilli-containing vaginal probiotics to cure or prevent bacterial or fungal vaginal dysbiosis: a systematic review and recommendations for future trial designs. BJOG 2020; 127: 287-299.
https://activemicrotechnologies.com/product/leucidal-liquid-sf/
⁴ Alex Gomelsky, Roger R. Dmochowsk “GAG Layer Replenishment Therapy for Recurrent Infectious Bladder Dysfunction, Current Bladder Dysfunction Reports, June 2012, Volume 7, Issue 2, pp 113-119
⁵ Trivedi M K, Nayak G, Patil S, Tallapragada R M, Mishra R (2015) influence Biofield Treatment on Physicochemical Properties of Hydroxyethyl Cellulose and Hydroxypropyl Cellulose. J Mol Pharm Org Process Res 3: 126.
⁶ Scientific premise reference for GAG enhancement
⁷ Ikeda S¹, Neyts J, Verma S, Wickramasinghe A, Mohan P, De Clercq E, Antimicrob Agents Chemother, 1994 February;38(2):256-9. In vitro and in vivo inhibition of ortho- and paramyxovirus infections by a new class of sulfonic acid polymers interacting with virus-cell binding and/or fusion.
⁸ Trivedi M K, Nayak G, Patil S, Tallapragada R M, Mishra R (2015) Influence of Biofield Treatment on Physicochemical Properties of Hydroxyethyl Cellulose and Hydroxypropyl Cellulose. J Mol Pharm Org Process Res 3: 126.
⁹ https://clinicaltrials.gov/ct2/show/NCT02130518
¹⁰ van de Wijgert JHHM, Verwijs M C. Lactobacilli-containing vaginal probiotics to cure or prevent bacterial or fungal vaginal dysbiosis: a systematic review and recommendations for future trial designs BJOG 2020; 127: 287-299.
¹¹ Toh S L, Lee B B Ryan S, et al. Probiotics [LGG-BB12 or RC14-GR1] versus placebo as prophylaxis for urinary tract infection in persons with spinal cord injury [ProSCIUTTU]: a randomized controlled trial. Spinal Cord. 2019;57(7):550-561. doi:10.1038/s41393-019-0251-y
¹² Stapleton A E, Au-Yeung M, Hooton T M, Fredricks D N, Roberts P L, Czaja C A, Yarova-Yarovaya Y, Fiedler T, Cox M, Stamm W E Randomized, placebo-controlled phase 2 trial of a Lactobacillus crispatus probiotic given intravaginally for prevention of recurrent urinary tract infection Clin Infect Dis. 2011 May; 52(10):1212-7.
¹³ Takashi Asahara, Koji Nomoto, Masaaki Watanuki, Teruo Yokokura “Antimicrobial Activity of Intraurethrally Administered Probiotic Lactobacillus caseu in a Murine Model of Escherichia coli Urinary Tract Infection Takashi Asahara, Koji Nomoto, Masaaki Watanuki, Teruo YokokuraAntimicrobial Agents and Chemotherapy June 2001, 45 (6)1751
¹⁴ Stapleton A E, Au-Yeung M, Hooton T M, Fredricks D N, Roberts P L, Czaja C A, Yarova-Yarovaya Y, Fiedler T, Cox M, Stamm W E Randomized, placebo-controlled phase 2 trial of a Lactobacillus crispatus probiotic given intravaginally for prevention of recurrent urinary tract infection Clin Infect Dis. 2011 May; 52(10):1212-7.
¹⁵ Borchert D, Sheridan L, Papatsoris A, Faruquz Z, Barua J M, Junaid Pati Y, Chinegwundoh F, Buchholz N. Prevention and treatment of urinary tract infection with probiotics. Review and research perspective. Indian J Urol 2008;24:139-44
¹⁶ https://www.slideshare.net/jitenpatel13/a-review-onhydroxyethyicellulose
¹⁷ Chaturvedi, Mayank, Manish Kumar, and Kamla Pathak. “A Review on Mucoadhesive Polymer Used in Nasal Drug Delivery System” Journal of Advanced Pharmaceutical Technology & Research 2.4 (2011): 215-222. PMC. Web. 10 Jan. 2018.
¹⁸ Gregor Reid, Dee Beuerman, Christine Heinemann, Andrew W. Bruce, Probiotic Lactobacillus dose required to restore and maintain a normal vaginal flora, FEMS Immunology & Medical Microbiology, Volume 32, Issue 1, December 2001, Pages 37-41
¹⁹ Borchert D, Sheridan L, Papatsoris A, Faruquz Z, Barua J M, Junaid I, Pati Y, Chinegwundoh F, Buchholz N. Prevention and treatment of urinary tract infection with probiotics: Review and research perspective. Indian J Urol 2008;24:139-44
²⁰ Akgül T, Karakan T. The role of probiotics in women with recurrent urinary tract infections. Turk J Urol. 2018;44(5):377-383. doi:10.5152/tud.2018.48742
²¹ Grin P M¹, Kowalewska P M, Alhazzan W, Fox-Robichaud A E. Lactobacillus for preventing recurrent urinary tract infections in women: meta-analysis, Can J Urol. 2013 February;20(1):6607-14.
²² Sadeghi-Bojd S, Naghshizadian R, Mazaheri M, Ghane Sharbaf F. Efficacy of probiotic prophylaxis after the first febrile urinary tract infection in children with normal urinary tracts [published online May 17, 2019]. J Pediatric Infect Dis Soc. doi:10.10934/jpids/piz025
²³ Probiotics to prevent the need for, and augment the use of, antibiotics. Reid GCan J Infect Dis Med Microbiol. 2006 September; 17(5):291-5.
²³ Akgül T, Karakan T. The role of probiotics in women with recurrent urinary tract infections. Turk J Urol. 2018;44(5):377-381 doi:10.5152/tud.2018.48742 (clinical trial review)
²⁵ Falagas M E, Betsi G I, Tokas T, Athanasiou S. Probiotics for prevention of recurrent urinary tract infections in women. Drugs. 2006;66(9):1253-61
²⁶ a. Reid G. Jass J, Sebulsky M T, McCormick J K. Potential uses of probiotics in clinical practice. Clinical Microbiol Rev. October 2003;16(4):658-672. [PMC free article] [PubMed] [Google Scholar]
b. De LeBlanc Ade M, Castillo N A, Perdigon G. Anti-infective mechanisms induced by a probiotic Lactobacillus strain against Salmonella enterica serovar Typhimurium infection. Int J Food Microbiol. 2010;138(3):223-231. 10.1016/j.ijfoodmicro.2010.01.020 [PubMed] [CrossRef] [Google Scholar]
c. Ramos A N, Cabral M E, Noseda D, Bosch A, Yantorno O M, Valdez J C. Antipathogenic properties of Lactobacillus plantarum on Pseudomonas aeruginosa; the potential use of its supernatants in the treatment of infected chronic wounds. Wound Repair Regen. 2012;20(4):552-562. 10.1111/j.1524-475X.2012.00798.x [PubMed] [CrossRef] [Google Scholar]
d. Valdez J C, Peral M C, Rachid M, Santana M, Perigon G. Interference of Lactobacillus plantarum with Pseudomonas aeruginosa in vitro and infected burns: the potential use probiotics in wound treatment. Clin Microbiol Infect. lune 2005;11(6):472-479. 10.1111/j.1469-0691.2005.01142.x
²⁷ https://www.mdlinx.com/surgery/article/1319 Probiotics accelerate wound healing in patients with diabetic foot ulcer
²⁸ Beerepoot M A, ter Riet G, Nys S et al: Lactobacilli vs antibiotics to prevent urinary tract infections: a randomized, double-blind, noninferiority trial in postmenopausal women. Arch Intern Med 2012; 172:704.
   a. Czaja C A, Stapleton A E, Yarova-Yarovaya Y et al: Phase I trial of a Lactobacillus crispatus vaginal suppository for prevention of recurrent urinary tract infection in women. Infect Dis Obstet Gynecol 2007; 2007: 35387.
   b. Reid G, Bruce A, Taylor M. Instillation of Lactobacillus and stimulation of indigenous organisms to prevent recurrence of urinary tract infections. Microecol Ther 1995; 32.
   c. Stapleton A E, Au-Yeung M, Hooton T M et al: Randomized, placebo-controlled phase 2 trial of a Lactobacillus crispatus probiotic given intravaginally for prevention of recurrent urinary tract infection. Clin Infect Dis 2011; 52: 1212.
²⁹ https://www.cochranelibrary.com/cdsr/doi/10.1002/14651858.CD008772.pub2/full#CD008772-sec1-0007
³⁰ Toh S L, Lee B B, Ryan S, et al. Probiotics [LGG-BB12 or RC14-GR1] versus placebo as prophylaxis for urinary tract infection in persons with spinal cord injury [ProSCIUTTU]: a randomized controlled trial. Spinal Cord. 2019;57(7):550-561. doi:10.1038/s41393-019-0251-y
³¹ Alain L. Servin, FEMS Microbiology Reviews 28 (2004) 405-440
³² Akgül T, Karakan T. The role of probiotics in women with recurrent urinary tract infections. Turk J Urol. 2018;44(5):377-383. doi:10.5152/tud.2018.48742
³³ Grin P M¹, Kowalewska P M, Alhazzan W, Fox-Robichaud A E. Lactobacillus for preventing recurrent urinary tract infections in women: meta-analysis., Can J Urol. 2013 February;20(1):6607-14.
³⁴ Sadeghi-Bojd S, Naghshizadiarian R, Mazaheri M, Ghane Sharbaf F, Assadi F. Efficacy of probiotic prophylaxis after the first febrile urinary tract infection in children with normal urinary tracts [published online May 17, 2019]. J Pediatric Infect Dis Soc. doi:10.1093/jpids/piz025
³⁵ Probiotics to prevent the need for, and augment the use of, antibiotics. Reid GCan J Infect Dis Med Microbiol. 2006 September; 117(5):291-5.
³⁶ Akgül T, Karakan T. The role of probiotics in women with recurrent urinary tract infections. Turk J Urol. 2018;44(5):377-383. doi:10.5152/tud.2018.48742 (clinical trial review)
³⁷ Falagas M E, Betsi G I, Tokas T, Athanasiou S. Probiotics for prevention of recurrent urinary tract infections in women. Drugs. 2006;66(9):1253-61
³⁸
   a. Reid G, Jass J, Sebulsky M T, McCormick J K. Potential uses of probiotics clinical practice. Clinical Microbiol Rev. October 2003;16(4):658-672. [PMC free article] [PubMed] [Google Scholar
   b. De LeBlanc Ade M, Castillo N A, Perdigon G. Anti-infective mechanisms induced by a probiotic Lactobacillus strain against Salmonella enterica serovar Typhimurium infection. Int J Food Microbiol. 2010;138(3):223-231. 10.1016/j,ijfoodmicro.2010.01.020 [PubMed] [CrossRef] [Google Scholar]
   c. Ramos A N, Cabral M E, Noseda D, Bosch A, Yantorno O M, Valdez J C. Antipathogenic properties of Lactobacillus plantarum on Pseudomonas aeruginosa: the potential use of its supernatants in the treatment of infected chronic wounds. Wound Repair Regen. 2012;20(4):552-562. 10.1111/j.1524-475X.2012,00798.x [PubMed] [CrossRef] [Google Scholar]
   d. Valdez J C, Feral M C, Rachid M, Santana M, Perigon G. Interference of Lactobacillus plantarum with Pseudomonas aeruginosa in vitro and infected burns: the potential use of probiotics in wound treatment. Clin Microbiol Infect. June 2005;11 (6):472-479. 10.1111/j.1469-0691.2005.01142.x [PubMed] [CrossRef] [Google Scholar
³⁹ https://www.mdlinx.com/surgery/article/1319 Probiotics accelerate wound healing in patients with diabetic foot ulcer
⁴⁰ Beerepoot M A, ter Riet G, Nys S et al: Lactobacilli vs antibiotics to prevent urinary tract infections: a randomized, double-blind, noninferiority trial in postmenopausal women. Arch Intern Med 2012; 172:704.
   Czaja C A, Stapleton A E, Yarova-Yarovaya Y et al: Phase I trial of a Lactobacillus crispatus vaginal suppository for prevention of recurrent urinary tract infection in women. Infect Dis Obstet Gynecol 2007; 2007: 35387.
   b. Reid G, Bruce A, Taylor M: Instillation Lactobacillus and stimulation of indigenous organisms to prevent recurrence of urinary tract infections. Microecol Ther 1995; 32.
   c. Stapleton A E, Au-Yeung M, Hooton T M et al: Randomized, placebo-controlled phase 2 trial of a Lactobacillus crispatus probiotic given intravaginally for prevention of recurrent urinary tract infection. Clin Infect Dis 2011; 52: 1212.
⁴¹ https://www.cochranelibrary.com/cdsr/doi/10.1002/14651858.CD008772.pub2/full#CD008772-sec1-0007
⁴² Beerepoot M, Geerlings S. Non-Antibiotic Prophylaxis for Urinary Tract Infections. Pathogens. 2016;5(2):36. Published 2016 Apr. 16. doi:10.3390/pathogens5020036
⁴³ D-mannose powder for prophylaxis of recurrent urinary tract infections in women: a randomized clinical trial. Kranjc̆ec B, Papes̆ D, Altarac S World J Urol. 2014 February; 32(1):79-84.
⁴⁴ https://www.cochrane.org/CD010723/RENAL_probiotics-preventing-urinary-tract-infections-people-bladder-dysfunction-after-nervous-system t is uncertain if probiotics prevent urine infections in people with bladder dysfunction after a nervous system injury. Further, robustly designed studies are necessary
⁴⁵ Agnieszka Torzewska and Antoni Różalski, Vol. 62, No 1/2015127-132, Biochemica polonica
⁴⁶ Sarangapani, S *, Katherine Cavedon, David Gage Journal of Biomedical Materials Research, Volume 29 Issue 10, Pages 1185-1191,1995

What is claimed is:

1. A gel composition for use as a barrier for microbial attachment and growth on a surface, crystal formation, and/or enhancement of mucosal barrier function, the composition comprising one or more water soluble anionic polymers at a concentration up to 2% w/w of the final gel, one or more gel forming polymers at a concentration up to 3% w/w of the gel, one or more live stabilized probiotic powders at a concentration not exceeding 200 billion cfu/gm of the gel, one or more non-live probiotic fermentates or derivatives at a concentration up to 11% w/w of the gel, one or more natural or synthetic surfactants at a concentration up to 0.5% w/w of the gel, potentiating antioxidants at up to 0.3% w/w of the gel, and/or one or more natural disaccharides, oligosaccharides, or polysaccharides, carbohydrates, and/or sugar alcohols, as growth promoters at up to 0.5% w/w of the gel.

2. The composition of claim 1 that is prepared as a quick-setting gel at room temperature, providing a semi-rigid gel like consistency within 10-30 minutes.

3. The composition of claim 1 that is prepared by dispersing the probiotic powder in a liquid medium followed by addition of a gelling agent comprising said gel forming polymer.

4. The composition of claim 1 that is suitable for application to a medical device, such as a catheter, or for application to skin or a mucosal surface of a body.

5. The composition of claim 1 that is in the form of a water-soluble gel, ointment, emulsion, foam, or cream.

6. The composition of claim 1, which is 3D printed in form of a pattern or design.

7. The composition of claim 1, wherein the gel forming polymer is selected from cellulose polymers, uncrosslinked polyvinyalcohol, cellulose-based polymers such as carboxy methyl cellulose, hydroxypropyl methylcellulose, polyacrylic acid polymers, plant-based polysaccharides, and combinations thereof.

8. The composition of claim 1, wherein the water-soluble anionic polymer is selected from sulfonated and carboxylated polymers that inhibit crystal formation of insoluble calcium or magnesium salts.

9. The water-based composition of claim 8, wherein the anionic water-soluble polymer provides a mucosal protective effect; and the water-based composition further comprising growth promoters for Lactobacillus comprising sugar alcohols including Mannitol, carbohydrates including D- Mannose, Sugars, or a combination thereof.

10. The composition of claim 1, further comprising a non-aqueous gel carrier selected from silicone-based oils, polymers, and biocompatible non-aqueous carriers such as oil, petroleum-based products, or lipids.

11. The composition of claim 1, wherein said probiotic powder has certified shelf life and potency, and viability assurance.

12. The composition of claim 1, wherein the composition is capable of serving as a barrier for attachment and growth of microbes selected from bacteria, yeast, and viral pathogens.

13. The composition of claim 1, wherein the composition is anti-inflammatory.

14. The composition of claim 1 in the form of a dry patch or printed textile, wherein the gel composition of claim 1 is formed in or on the patch or textile upon contact with water.

15. A kit of parts comprising two or more pre-packaged components capable of forming a gel, cream, or ointment comprising the gel composition of claim 1 when the components are combined; and instructions for use.

16. The kit of parts of claim 15, wherein one or more pre-packaged components are in pods or capsules, wherein the components are capable of forming the gel, cream, or ointment upon the addition of water to the pods or capsules.

17. A method for preparing a prophylactic gel, ointment, or cream composition, the composition suitable for use as a barrier for microbial attachment and growth, crystal formation, and/or enhancement of mucosal barrier function, the method comprising combining one or more hydrophilic muco-adhesive agents and one or more non-aqueous carriers.

18. A method to aid in treating or preventing an infection, the method comprising administering to s subject in need thereof the gel composition of claim 1.

19. The method of claim 18, comprising administering the gel composition via a soluble suppository or pellet.

20. The method of claim 18, comprising implanting in the subject's body a medical device comprising or consisting of the composition of claim 1.
